(19) United States
(12) Patent Application Publication
(10) Pub. No.: US 2007/0116749 A1
(43) Pub. Date: May 24, 2007

(54) Method for treatment of cellulitis

(76) Inventor: Leonard D. Grossman, Brooklyn, NY (US)

Correspondence Address: Svetlana Short;NY Center for Plastic Surgery, 3039 Emmons Av, Brooklyn, NY, 11235, US

(21) Appl. No.: 11/285,648

(22) Filed: Nov. 21, 2005

(51) Int. Cl. A61L 15/00; A61K 31/045

(52) U.S. Cl. 424/445; 514/724

(57) ABSTRACT

A method of treating a patient having a skin infection comprises steps of: (i) covering the area of afflicted with infection with a dressing including an absorbent layer soaked in alcohol, such that the alcohol is in contact with the area of skin afflicted with infection; and (ii) keeping said absorbent layer soaked in alcohol in contact with the area afflicted of infection for at least 1 hour.

### CROSS-REFERENCE TO RELATED APPLICATIONS

This application relates to the application entitled “A Dressing for Treatment of Cellulitis” by Leonard Grossman, which is incorporated by reference herein and is filed concurrently herewith.

### BACKGROUND OF THE INVENTION

1. Field of the Invention

The present invention relates generally to a device and process for treatment of cellulitis, and other skin related bacterial infections.

2. Technical Background

Cellulitis and other skin related bacterial infections can result in severe trauma and suffering to the patient in the form of physical pain, inability to move the involved extremity, swelling, generalized spread of infection leading to limb loss and sometimes loss of a life. Other then in healthy individuals, these conditions are more frequently encountered in people with one or another form of diabetes, peripheral vascular disorders and some form of immunological deficiency.

Generally, early stage of cellulitis is treated with oral antibiotics and extremity rest. In a more serious situation, such as widespread cellulitis in a person with one or more of the medical problems mentioned above, the patient is usually admitted to the hospital for administration of intravenous antibiotics, many days of medical observation by the doctors and nursing staff, extremity rest and elevation and combination of moist heat. All of this results in a high cost of the medical facility, doctors and nursing fees, high cost of the intravenous antibiotics and time lost from work by the patient.

### SUMMARY OF THE INVENTION

According to one aspect of the present invention,

A method of treating cellulitis comprises) covering the area of afflicted by cellulitis with a dressing that includes alcohol, for an effective amount of time to inhibit or prevent cell injury due to cellulites.

According to one embodiment a method of treating a patient having a skin infection comprises steps of:
(i) covering the area of afflicted with infection with a dressing including an absorbent layer soaked in alcohol, such that the alcohol is in contact with the area of skin afflicted with infection; and
(ii) keeping the absorbent layer soaked in alcohol in contact with the area afflicted with infection for at least 1 hour.

According to some embodiments of the present invention, alcohol is kept in contact with the area afflicted with infection for at least 2, and preferably at least 4 hours.

According to one aspect of the present invention a method of treating cellulites comprises: covering the area of afflicted by cellulitis with a dressing including alcohol, for an effective amount of time to inhibit or prevent cell injury due to cellulites.

According to one embodiment of the present invention a method of treating a patient having skin afflicted with infection comprises:
(i) covering the area of skin afflicted with infection with an absorbent layer soaked in alcohol such that said alcohol is in contact with the area of skin afflicted with infection;
(ii) covering said absorbent layer soaked in alcohol with a layer of alcohol impervious material;
(iii) covering said alcohol impervious material with a layer of an insulating material;
(iv) keeping said absorbent layer soaked in alcohol in contact with the area afflicted with infection for at least 2 hours.

According to some embodiments the skin afflicted with infection has cellulites. It is an advantage of the present invention that an inexpensive dressing, which when applied locally to an area of cellulitis will treat, control and cure the infection in a matter of hours.

Additional features and advantages of the invention will be set forth in the detailed description which follows, and in part will be readily apparent to those skilled in the art from that description or recognized by practicing the invention as described herein, including the detailed description which follows, the claims, as well as the appended drawings.

It is to be understood that both the foregoing general description and the following detailed description present embodiments of the invention, and are intended to provide an overview or framework for understanding the nature and character of the invention as it is claimed. The accompanying drawings are included to provide a further understanding of the invention, and are incorporated into and constitute a part of this specification. The drawings illustrate various embodiments of the invention, and together with the description serve to explain the principles and operations of the invention.

### BRIEF DESCRIPTION OF THE DRAWINGS

FIG. 1 is a flowchart depicting one embodiment of the present invention;

FIG. 2 is a flowchart depicting another embodiment of the present invention;

FIG. 3 is a schematic drawing of a patient with a dressing according to an embodiment of the present invention; and

FIG. 4 is a schematic side view of a dressing according one embodiment of the present invention.

### DETAILED DESCRIPTION OF THE PREFERRED EMBODIMENTS

Reference will now be made in detail to the present preferred embodiments of the invention, examples of which are illustrated in the accompanying drawings. Whenever possible, the same reference numerals will be used throughout the drawings to refer to the same or like parts.

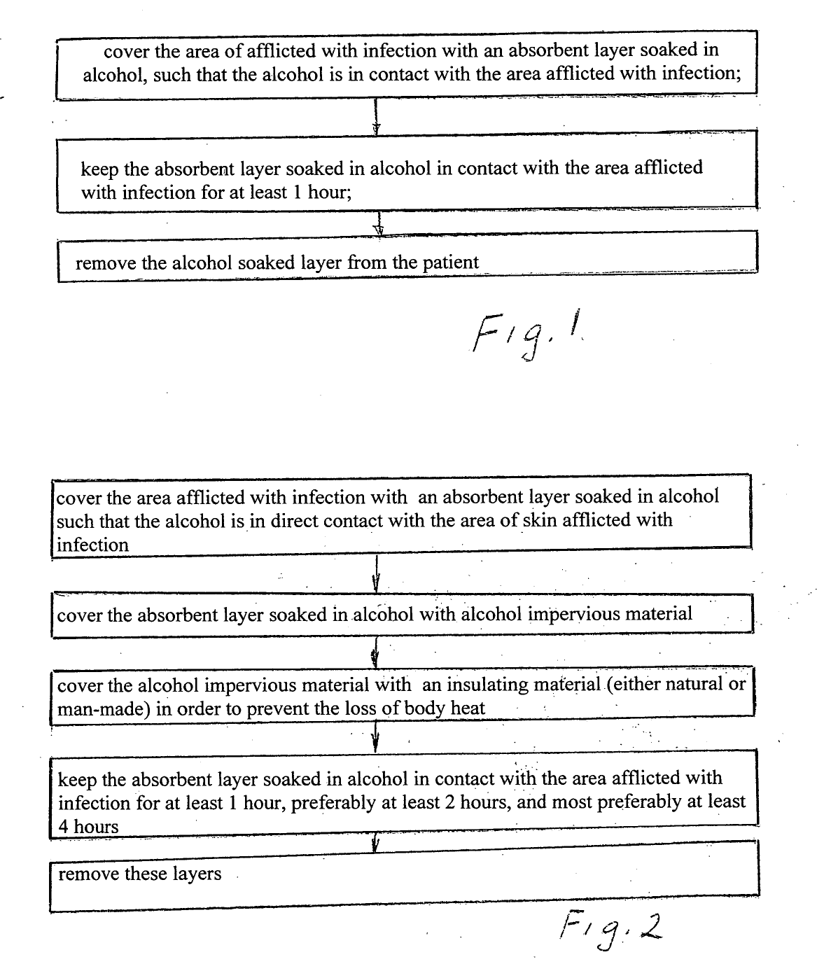

As shown in FIG. 1, a method of treating a patient afflicted with skin infection includes the steps of: (i) covering the area of afflicted with infection with an absorbent layer soaked in alcohol, such that the alcohol is in contact with the area afflicted with infection; (ii) keeping the absorbent layer soaked in alcohol in contact with the area afflicted with infection for at least 1 hour; and (iii) removing the alcohol soaked layer from the patient. Examples of infections treatable by this method are cellulites, folliculitis, furunculitis, or other infectious conditions related to skin.

According to some embodiments of the present invention, alcohol is kept in contact with the area afflicted with infection for at least for and preferably for at least 4 hours.

As shown in FIG. 2, according to one embodiment of the present invention a method of treating a patient having skin afflicted with infection comprises: (i) covering the area afflicted with infection with an absorbent layer soaked in alcohol such that the alcohol is in direct contact with the area of skin afflicted with infection; (ii) covering the absorbent layer soaked in alcohol with alcohol impervious material, such as cellophane, for example; (iii) covering the alcohol impervious material with an insulating material (either natural or man-made) in order to prevent the loss of body heat; (iv) keeping the absorbent layer soaked in alcohol in contact with the area afflicted with infection for at least 1 hour, preferably at least 2 hours, and most preferably at least 4 hours; and (v) removing these layers. The dressing may be kept in contact with the skin for 3-7 hours or even longer (for example 6-10 hours), depending on the needs of the individual.

I believe that this method works because alcohol permeates through the porous skin layers to the infected site, where it kills the infection causing bacteria directly. Although the continuous exposure to alcohol for several hours will dry the skin, and may form patches of dry skin, the dried skin cells will flake off within a few days or weeks. Because the alcohol is in contact with the infected area for a relatively long period of time and because it penetrates through the skin, it is preferable to use ethanol (drinking alcohol) if the infected area is relatively large (for example, more than 2 cm²). Typical application duration is 3 to 7 hours, which may be repeated after 10 to 12 hours. Of course, one longer application is equivalent to several shorter applications. For example, a fresh dressing may be applied for one hour three times within a 12 hour period, which, due to a larger initial alcohol absorption, will produce a result similar to that produced by a dressing that is applied for about 2.5 hours.

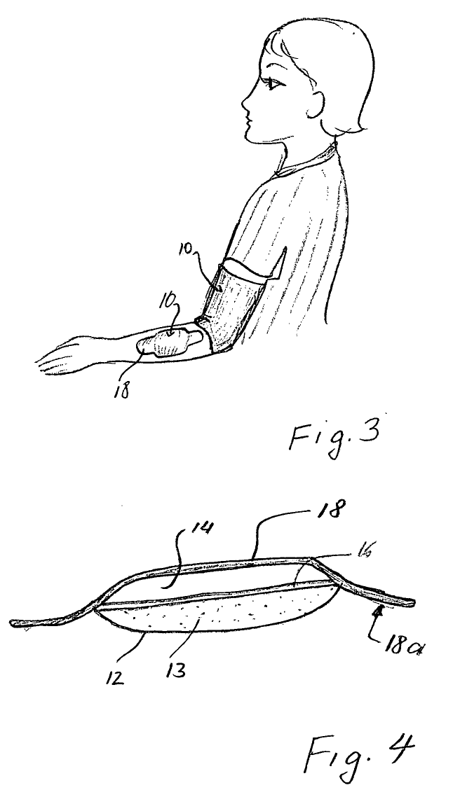

According to one embodiment of the present invention a simple, inexpensive dressing 10 containing a layer 12 of absorbent material presoaked in alcohol 13 is applied to the skin area afflicted with infection for several hours, taken off, and a new dressing 10 is applied again in 10-12 hours, if needed. FIG. 3 illustrates schematically a person with such dressing 10. One embodiment of the dressing according to the present invention is shown in FIG. 4, and is designated generally throughout by the reference numeral 10.

According to one embodiment of the present invention the dressing 10 comprises at least three layers:

1. A layer 12 of alcohol (for example, ethanol or isopropyl alcohol) soaked absorbent material, for example a layer of cotton. The material may be either natural or synthetic. This layer 12 will come in direct contact with the area of skin afflicted with infection, as determined by visual inspection. Alternatively, alcohol may be kept in contact with the skin by any other other means.

2. An occlusive layer 14 (alcohol impervious layer) to prevent or to slow down evaporation of alcohol. For example, a thin layer cellophane, polyethylene plastic or polyethylene, such as clingwrap® available from Glad Products Company of Oakland, Calif. is a suitable alcohol impervious material. This layer 14 will limit evaporation of alcohol and prolong the effect of alcohol absorption.

3. Another layer 16 of an insulating material, for example cotton wool. The material for the insulating layer may also be a synthetic material. This layer 16 will isolate and prevent the loss of body heat from the area being treated.

The dressing 10 should not interfere with circulation, either venous or arterial and should not compress an extremity in a circumferential manner. The dressing may be optionally kept in place in a tube of an elastic mesh, for example BandNet™ available from Western Medical Limited of Tenafly, N.J. The dressing may also kept in place by a sticky section(s) 18a of the outside wrap 18, similar to that of small bandages, as long as the infected area is fully covered. The dressing 10 may come in various shape and sizes.

The treatment results in acute improvement of the condition, which can be seen visually, for example, in as little as 8 hours, in some cases with complete resolution of the symptoms in the same amount of time. The treatment can be performed at home as instructed by the medical personal, or the patient, who is aware of the condition, especially in the early stages of infection.

The application of dressing can be repeated every 12 hours, but the application itself should be, for example, about six to eight hours long.

This dressing could be applied just like a Band-Aid®, may come in different sizes and shapes (for example, rectangular, circular, or oval) and may be pretreated with alcohol and ready to use out of a pack. Alternatively it may be provided w/o the alcohol, and the end user may apply the alcohol to the layer 12, prior to using the dressing. The dressing can also be customized to any size when needed. The dressing is intended for the treatment of relatively superficial infections (for example, cellulitis), even though some abscesses respond to this treatment very well also.

In accordance with the one embodiment of the present invention, the present invention for a method of treating a patient having skin afflicted with infection, includes the step of covering the area of skin afflicted with infection with a dressing comprising an absorbent layer soaked in alcohol such that said alcohol is in contact with the area of cellulitis and keeping the alcohol soaked layer in contact with the area of skin afflicted with infection for at least 1 hour. The term “skin afflicted by infection” is defined herein as the skin area having cellulitis, redness, red streaks, or other indication(s) of infection or abscess.

It will be apparent to those skilled in the art that various modifications and variations can be made to the present invention without departing from the spirit and scope of the invention. Thus it is intended that the present invention cover the modifications and variations of this invention provided they come within the scope of the appended claims and their equivalents.

What is claimed is:

1. A method of treating a patient affected with cellulitis, said method comprising steps of: (i) covering the area of cellulitis with a dressing comprising an absorbent layer soaked in alcohol such that said alcohol is in contact with the area of cellulitis; (ii) keeping said alcohol soaked layer in contact with the area of cellulitis for at least 1 hour; and (iii) removing said layer.

2. The method according to claim 1, wherein said absorbent layer soaked in alcohol is kept in contact with the area of cellulitis for at least 4 hours.

3. The method according to claim 1, wherein said absorbent layer soaked in alcohol is kept in contact with the area of cellulitis for 6 to 10 hours.

4. The method according to claim 2, wherein alcohol is ethanol.

5. The method according to claim 2, wherein alcohol is isopropyl alcohol.

6. A method of treating a patient affected with cellulitis, said method comprising:
(I) covering the area of cellulitis with a dressing comprising
a) an absorbent layer soaked in alcohol such that said alcohol is in contact with the area of cellulitis;
b) covering said absorbent layer soaked in alcohol with alcohol impervious material;
c) covering said alcohol impervious material with an insulating material;
d) keeping said absorbent layer soaked in alcohol in contact with the area of cellulitis for at least 1 hour; and
(II) removing said dressing.

7. The method according to claim 6, wherein alcohol is ethanol.

8. The method according to claim 6, wherein alcohol is isopropyl alcohol.

9. The method according to claim 6, wherein said alcohol impervious layer is cellophane.

10. The method according to claim 6, wherein said insulating material includes cotton or wool.

11. The method according to claim 6, wherein said absorbent layer soaked in alcohol is kept in contact with the area of cellulitis for at least 4 hours.

12. The method according to claim 11, wherein alcohol is ethanol.

13. The method according to claim 11, wherein alcohol is isopropyl alcohol.

14. The method according to claim 11, wherein said absorbent layer soaked in alcohol is kept in contact with the area of cellulitis for 6 to 10 hours.

15. A method of treating a patient having skin afflicted with infection, said method comprising:
a) covering the area afflicted with infection with an absorbent layer soaked in alcohol such that said alcohol is in contact with the area afflicted with infection;
b) covering said absorbent layer soaked in alcohol with alcohol impervious material;
c) covering said alcohol impervious material with an insulating material;
d) keeping said absorbent layer soaked in alcohol in contact with the area afflicted with infection for at least 1 hour; and
e) removing said layers.

16. The method according to claim 15, wherein alcohol is ethanol.

17. The method according to claim 15, wherein alcohol is isopropyl alcohol.

18. The method according to claim 15, wherein said alcohol impervious layer is cellophane.

19. The method according to claim 15, wherein said insulating material includes cotton or wool.

20. The method according to claim 6, wherein said absorbent layer soaked in alcohol is kept in contact with the area of cellulitis for at least 4 hours.

21. The method according to claim 20, wherein alcohol is ethanol.

22. The method according to claim 21, wherein alcohol is isopropyl alcohol.

23. The method according to claim 11, wherein said absorbent layer soaked in alcohol is kept in contact with the area afflicted with infection for 6 to 10 hours.

24. A method of treating a patient having a skin infection comprises steps of:
(i) covering the area of afflicted with infection with a dressing including an absorbent layer soaked in alcohol, such that the alcohol is in contact with the area of skin afflicted with infection; and
(ii) keeping the absorbent layer soaked in alcohol in contact with the area afflicted with infection for at least 1 hour.

25. The method of claim 24 wherein said absorbent layer is kept in with the area afflicted with infection for at 3 to 7 hours.

26. A method of treating cellulitis comprising:
covering the area of afflicted by cellulitis with a dressing that includes alcohol, for an effective amount of time to inhibit or prevent cell injury due to cellulites.
